(19) United States
(12) Patent Application Publication
(10) Pub. No.: US 2023/0212697 A1
(43) Pub. Date: Jul. 6, 2023

(54) ISOTHERMAL REAL-TIME PCR METHOD FOR DETERMINING PRESENCE OF A PRE-DETERMINED NUCLEIC ACID SEQUENCE OF A BACTERIUM OF THE MOLLICUTES CLASS IN A SAMPLE

(71) Applicant: CERTUS MOLECULAR DIAGNOSTICS AG, Bern (CH)

(72) Inventors: Samuel ZÜRCHER, Bern (CH); Alexander LÜTHI, Bern (CH); Lea WEIBEL, Bern (CH)

(21) Appl. No.: 18/008,809

(22) PCT Filed: Jun. 9, 2021
(86) PCT No.: PCT/EP2021/065548

§ 371 Date: Dec. 7, 2022

(30) Foreign Application Priority Data
Jun. 9, 2020 (EP) 20179107.6

(51) Int. Cl. C12Q 1/689; C12Q 1/686

(52) U.S. Cl. CPC C12Q 1/689; C12Q 1/686; C12Q 2600/16

(57) ABSTRACT

The present invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether a double-stranded elongated DNA sequence is present in the sample, wherein presence of the double-stranded elongated DNA sequence in the sample is indicative of the presence of the pre-determined nucleic acid sequence in the sample, wherein the pre-determined nucleic acid sequence is of a bacterium of the Mollicutes class and wherein no F3 primer is used.

### RELATED APPLICATIONS

The instant application is a 35 U.S.C. § 371 filing of International Patent Application No. PCT/EP2021/065548, filed Jun. 9, 2021, which claims priority to European Patent Application No. 20179107.6, filed Jun. 9, 2020, the entire contents of which are incorporated herein by reference for all purposes.

### SEQUENCE LISTING

The instant application contains a Sequence Listing which has been submitted electronically in ASCII format and is hereby incorporated by reference in its entirety. Said ASCII copy, created on Dec. 6, 2022, is named 737568_VSO9-005US_ST25.txt and is 3,515 bytes in size.

The present invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether a double-stranded elongated DNA sequence is present in the sample, wherein presence of the double-stranded elongated DNA sequence in the sample is indicative of the presence of the pre-determined nucleic acid sequence in the sample, wherein the pre-determined nucleic acid sequence is of a bacterium of the Mollicutes class and wherein no F3 primer is used.

Contamination of cell lines and primary cells in research laboratories as well as cell-derived products used or manufactured in biotechnology or the pharma industry with Mollicutes is a major problem. Mollicutes are bacteria of various animals, including humans, and plants. They live in or on the host's cells and many are disease causing. Due to their abundance, cell or tissue cultures and other products used and manufactured in industries dealing with cells/tissues get easily contaminated. Such contaminated materials have to be decontaminated or destroyed, causing additional costs and burden. The earlier a contamination can be detected, the easier, less costly and faster it is to get the contamination under control, i.e. decontaminate the material.

In accordance with the above, there is a need for reliable, cost-efficient and fast methods for determining presence of bacteria of the Mollicutes class.

The above technical problem is solved by the embodiments provided herein and as characterized in the claims.

Accordingly, the present invention relates to, inter alia, the following embodiments.
1. A method for determining presence of a pre-determined nucleic acid sequence in a sample, the method comprising the steps of:
   (a) adding one or more enzyme(s) providing activities of RNA- and/or DNA-dependent DNA polymerase activity and strand-displacement activity to the sample to be analysed for the presence of the pre-determined nucleic acid sequence;
   (b) adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence;
   (c) incubating the sample resulting from steps (a) and (b) at a fixed temperature;
   (d) determining whether a double-stranded elongated DNA sequence is present in the sample,
      wherein presence of the double-stranded elongated DNA sequence in the sample is indicative of the presence of the pre-determined nucleic acid sequence in the sample
   wherein the pre-determined nucleic acid sequence is of a bacterium of the Mollicutes class and wherein no F3 primer is used.
2. The method of embodiment 1, wherein the at least five primers comprise a forward inner primer (FIP), backward inner primer (BIP), loop primer forward (LPF) and loop primer backwards (LPB), respectively.
3. The method of embodiment 1 or 2, wherein the at least five primers further comprise a B3 primer.
4. The method of any one of embodiments 1 to 3, wherein the pre-determined nucleic acid sequence is an RNA or DNA sequence.
5. The method of any one of embodiments 1 to 4, wherein the bacterium of the Mollicutes class is of the genus Mycoplasma, Spiroplasma, Acholeplasma, or Ureaplasma.
6. The method of embodiment 5, wherein the bacterium is M. orale, M. arginini, M. fermentans, M. hyorhinis, A. laidlawii, M. hominis, M. synoviae, S. citri, M. pneumoniae, M. bovis, M. salivarium or M. gallisepticum.
7. The method of any one of embodiments 4 to 6, wherein the RNA is comprised in 16S rRNA or 23S rRNA.
8. The method of any one of embodiments 4 to 6, wherein the DNA is the gene coding for 16S rRNA or the gene coding for 23S rRNA.
9. The method of any one of embodiments 1 to 8, wherein the sample is obtained from primary- or modified cells and/or tissues, cell cultures, culture medium and/or additives, cell derived products, laboratory equipment or biopharmaceutical products such as advanced therapy medicinal products (ATMPs).
10. The method of any one of embodiments 1 to 9, wherein the fixed temperature is between 50 and 75° C.
11. The method of any one of embodiments 1 to 10, wherein the sample in step (c) is incubated for 1 to 120 minutes.
12. The method of any one of embodiments 1 to 11, wherein presence of the double-stranded elongated DNA sequence in the sample is determined by using a nucleic acid molecule hybridisable to the double-stranded elongated DNA sequence, in particular wherein the nucleic acid molecule is labelled, using a molecule that intercalates in the double-stranded elongated DNA sequence or using turbidity measurement.
13. A method of decontaminating a cell and/or tissue culture contaminated by a bacterium of the Mollicutes class, the method comprising administering to the culture an efficient amount of an antibiotic, wherein the culture has previously been determined to be infected by a bacterium of the Mollicutes class using the method of any one of embodiments 1 to 12.
14. The method of embodiment 13, wherein the antibiotic drug is BM Cyclin.

Accordingly, in a first aspect, the invention relates to a method for determining presence of a pre-determined nucleic acid sequence in a sample, the method comprising the steps of adding one or more enzyme(s) providing activities of RNA- and/or DNA-dependent DNA polymerase activity and strand-displacement activity to the sample to be analysed for the presence of the pre-determined nucleic acid sequence; adding at least five DNA primers to the sample to be analysed for the presence of the pre-determined nucleic acid sequence, wherein at least one DNA primer comprises a sequence hybridisable to the nucleic acid sequence and at least one DNA primer comprises a sequence hybridisable to the DNA sequence reverse-complementary to the nucleic acid sequence; incubating the sample resulting at a fixed temperature; determining whether a double-stranded elongated DNA sequence is present in the sample, wherein presence of the double-stranded elongated DNA sequence in the sample is indicative of the presence of the pre-determined nucleic acid sequence in the sample, wherein the pre-determined nucleic acid sequence is of a bacterium of the Mollicutes class.

The term “pre-determined nucleic acid sequence”, as used herein, refers to a nucleic acid sequence, preferably an RNA or DNA sequence, where the skilled person is aware that it is part of a bacterium of the Mollicutes class. In particular, the pre-determined nucleic acid sequence, within the present invention, is a sequence that is detectable using the method of the present invention. That is, a nucleic acid sequence available to the skilled person is pre-determined if the skilled person can determine whether the sequence will likely be detectable in a sample using the methods as provided herein. Within the present invention, the pre-determined nucleic acid sequence comprises at least one primer binding site that is at least partially identical to at least one of the primers used in the methods of the invention. Primer binding sites are considered identical to a primer site if the sequence is exactly identical or if they differ only in that one sequence comprises uracil instead of thymidine and/or if they differ only in that one sequence comprises one or more modified nucleotides instead of the respective non-modified nucleotide(s). The pre-determined nucleic acid sequence of a bacterium of the Mollicutes class can be from any part of the bacterium. In some embodiments, the pre-determined nucleic acid sequence of a bacterium of the Mollicutes class is a sequence where the skilled person is aware that it is part of at least one part of the bacterium selected from the group of nucleus, ribosome, mitochondria, cytoplasm and plasmid.

The term “sample”, as used herein, refers to any specimen potentially comprising the pre-determined nucleic acid sequence of a bacterium of the Mollicutes class. A sample as used in the methods of the present invention can be derived from primary- or modified cells, tissues, cell cultures, culture medium, additives, cell derived products, laboratory equipment, biopharmaceutical products (such as ATMPs), blood, a living body (e.g., a plant, and/or an animal), microorganisms and/or, e.g., samples separated from food, soil and/or waste water. Isolation of nucleic acid from the initial sample can be carried out by any method known to the person skilled in the art, such as, e.g., lysis treatment with a surfactant, sonic treatment, shaking agitation using glass beads or a French press method. In the methods of the present invention, an endogenous nuclease may be used to reduce the length of nucleic acid molecules. In the methods of the present invention, purification of the nucleic acid may be performed by, for example, phenol extraction, chromatography, ion exchange, gel electrophoresis, density-dependent centrifugation and/or other methods known to the person skilled in the art.

The terms “DNA primer” or “primer”, as used herein, refer to a nucleic acid molecule comprising a 3′-terminal —OH group that, upon hybridisation to a complementary nucleic acid sequence, can be elongated, e.g., via an enzymatic nucleic acid replication reaction. The primer set according to the present invention is used for amplification of nucleic acids, that is, for a LAMP analysis or a RT-LAMP analysis. Both the upper and lower limits of the length of the primer are empirically determined. The primer described herein can be a forward primer or a reverse primer. The term “backward primer”, as used herein, refers to a primer priming the antisense strand of a DNA sequence to allow the polymerase to extend in one direction along the complementary strand of a DNA sequence. At least one backward primer also serves as the RT primer for reverse transcription. The term “forward primer”, as used herein, refers to a primer priming the sense strand of a DNA sequence to allow a polymerase to extend in one direction along one strand of a DNA sequence.

An enzyme providing activities of RNA- and/or DNA-dependent DNA polymerase activity can synthesize DNA in the 5′->3′ direction based on a template composed of a DNA or RNA strand. As the skilled person is aware, such an enzyme will be successively adding nucleotides to the free 3′-hydroxyl group of the template. In this regard, the template strand determines the sequence of the added nucleotides based on Watson-Crick base pairing. The activity of the DNA polymerase may be RNA- and/or DNA-dependent. Exemplary polymerases include, but are not limited to Bst DNA polymerase, Vent DNA polymerase, Vent (exo-) DNA polymerase, Deep Vent DNA polymerase, Deep Vent (exo-) DNA polymerase, Bca (exo-) DNA polymerase, DNA polymerase I Klenow fragment, D29 phage DNA polymerase, Z-Taq™ DNA polymerase, ThermoPhi polymerase, 9° Nm DNA polymerase, and KOD DNA polymerase. See, e.g., U.S. Pat. Nos. 5,814,506; 5,210,036; 5,500,363; 5,352,778; and 5,834,285; Nishioka, M., et al. (2001) J. Biotechnol. 88, 141; Takagi, M., et al. (1997) Appl. Environ. Microbiol. 63, 4504.

As an enzyme providing activities of RNA-dependent DNA polymerase activity any suitable reverse transcriptase may be employed. In this regard, the enzyme to be used is not particularly limited, with the proviso that it has the activity to synthesize cDNA using RNA as the template. In addition, a substance which improves heat resistance of the nucleic acid amplification enzyme, such as trehalose, can be added.

When simply expressed as “5′-end side” or “3′-end side” in this specification, it means the direction in the chain which is regarded as the template in all cases. Also, when described that the 3′-end side becomes the starting point of complementary chain synthesis, it means that the 3′-end side —OH group is the starting point of complementary chain synthesis.

The term “strand displacement”, as used herein, refers to the ability of an enzyme to separate the DNA and/or RNA strands in a double-stranded DNA molecule and/or in a double-stranded RNA molecule during primer-initiated synthesis.

The term “hybridisation”, as used herein, refers to the annealing of complementary nucleic acid molecules. When two nucleic acids “hybridise to” each other, or when one nucleic acid “hybridises to” another, the two nucleic acid molecules exhibit a sufficient number of complementary nucleobases that the two nucleic acid molecules can anneal to each other under the particular conditions (e.g., temperature, salt and other buffer conditions) being utilized for a particular reaction. The most common mechanism of hybridisation involves hydrogen bonding (e.g., Watson-Crick, Hoogsteen or reversed Hoogsteen hydrogen bonding) between complementary nucleobases of the nucleic acid molecules. Hybridisation can occur under varying conditions. Stringent conditions are sequence-dependent and are determined by the nature and composition of the nucleic acid molecules to be hybridised. Nucleic acid hybridisation techniques and conditions are known to the skilled artisan and have been described extensively. See, e.g., Sambrook et al, Molecular Cloning: A Laboratory Manual 2nd ed. Cold Spring Harbor Press, 1989; Ausubel et al, 1987, Current Protocols in Molecular Biology; Greene Publishing and Wiley-Interscience, New York; Tijessen, 1993, Hybridization with Nucleic Acid Probes, Elsevier Science Publishers, B.V.; and Kricka, 1992, Non-Isotopic DNA Probe Techniques, Academic Press, San Diego, Calif.

The term “F3”, as used herein, refers to the outer forward primer of a primer set.

While previous LAMP methods assumed that the F3 primer is required for releasing a cDNA strand during the amplification process (see e.g. Nagamine et al. 2002. Molecular and Cellular Probes 16. 223-229), the inventors found that omission of the F3 primer does not impair the amplification process.

Within the present invention, it was surprisingly found that a five-primer system, wherein the F3 primer is omitted, is most efficient in detecting a pre-determined nucleic acid sequence. “Most efficient” as used herein means that detection is as fast and sensitive than commonly used techniques but maintains reliability, which is a prerequisite in tests used for detecting nucleic acids such as nucleic acids derived from Mollicutes. In addition, it was found that by using five primers instead of six primers as in the standard LAMP technology, shorter target sequences can be detected.

The invention provides a sample containing a pre-determined nucleic acid sequence, and a method for amplifying a nucleic acid, which comprises carrying out an amplification reaction of the pre-determined nucleic acid sequence in the sample, in a reaction system wherein at least one primer of the invention is present. In certain embodiments of the invention, at least one species of the primers is used in the nucleic acid amplification reaction of the invention. That is, the DNA primer described herein may be used in combination with other primers, or two species of the DNA primer described herein may be used.

It is preferred within the methods of the present invention that five DNA primers are used.

In some embodiments, the at least two of the primers employed in the invention are loop primers.

The term “loop primer”, as used herein, refers to a DNA primer comprising a sequence that is hybridisable to at least one loop region of an amplification product of the pre-determined RNA sequence. The loop region is formed by the annealing of a strand of an amplification product to itself. Typically, loop primers hybridise to generated DNA sequences and provide an increased number of starting points for the initiation of further DNA elongation processes. The use of loop primer can accelerate the amplification process.

Within the present invention, it is preferred that the at least five primers comprise a forward inner primer (FIP), backward inner primer (BIP), loop primer forward (LPF) and loop primer backwards (LPB), respectively.

The term “FIP” or “forward inner primer”, as used herein, refers to a forward primer that comprises a sequence for strand initiation and a sequence hybridisable to the same FIP-initiated strand.

The term “BIP” or “backward inner primer”, as used herein, refers to a backward primer that comprises a sequence for strand initiation and a sequence hybridisable to the same BIP-initiated strand.

The term “loop primer forward” or “LPF”, as used herein, refers to a loop primer that is a forward primer.

The term “loop primer backwards” or “LPB”, as used herein, refers to a loop primer that is a backwards primer.

Preferably, the at least five primers further comprise a B3 primer.

The term “B3”, as used herein, refers to the outer backward primer of a primer set.

The DNA Primer described herein that specifically binds to a target nucleic acid or its complementary sequence may be at least 10, 15, or 18 nucleotides in length, at least 18, 20, 22 or 24 nucleotides for B3, at least 25, 30, 33, or 36 nucleotides for FIP and BIP, and at least 10, 15, 17, or 18 for LPF and LPB. DNA Primers that specifically bind to a target nucleic acid sequence may have a nucleic acid sequence at least 80% complementarity, particularly 90% complementarity, more particularly 95%, 96%, 97%, 98%, 99% or 100% complementarity with the corresponding region.

These terms are commonly used in methods related to loop-mediated isothermal amplification (LAMP) methods, such as those described by Nagamine et al. 2002. Molecular and Cellular Probes 16. 223-229.

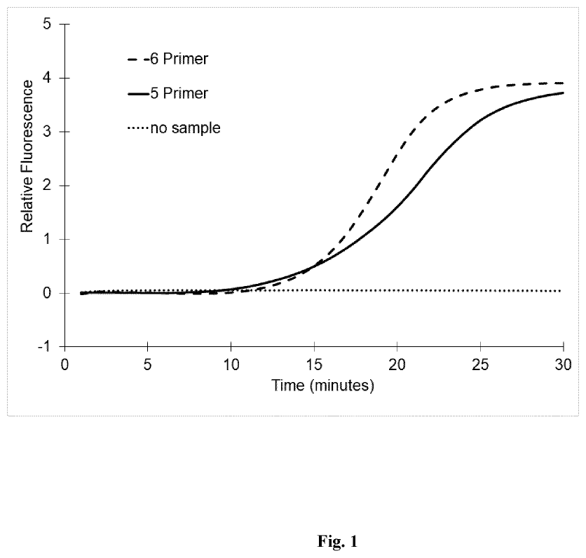

Within the methods of the present invention no F3 primer is used and it is thus preferred that the fifth primer is a B3 primer. This is because it was surprisingly found by the inventors that in the presence of a B3 primer but absence of an F3 primer, detection is faster and more sensitive. Using the methods of the present invention, detection was observed to be possible within ten minutes and more sensitive to detect a low number of pre-determined RNA sequence in a sample. That is, as it is shown in the appended Example, a positive detection of a pre-determined sequence of a Mycoplasma strain was achieved using five primers, in particular FIP, BIP, LPF, LPB and B3, within ten minutes after addition of primers and enzymes (FIG. 1). Accordingly, the methods of the present invention, for the first time, provide a reliable and fast way to detect a contamination with mollicutes using a five primer isothermal amplification system.

Within the methods of the present invention, one or more enzyme(s) providing activities of RNA- and/or DNA-dependent DNA polymerase activity and strand-displacement activity are used. That is, in case of an RNA sequence, all three activities are to be added to the RNA sequence to be analyzed. In case of a DNA sequence, activity of the RNA-dependent DNA polymerase is not required. The activities can be provided by one enzyme having all two/three activities, or several enzymes each having one or more of the two/three activities.

It is preferred that the pre-determined nucleic acid sequence is an RNA or DNA sequence.

Further, it is preferred that the bacterium of the Mollicutes class is of the genus Mycoplasma, Spiroplasma, Acholeplasma, or Ureaplasma. Most preferably, the bacterium is of the genus Mycoplasma.

It is further preferred that the bacterium is M. orale, M. arginini, M. fermentans, M. hyorhinis, A. laidlawii, M. hominis, M. synoviae, S. citri, M. pneumoniae, M. bovis, M. salivarium or M. gallisepticum.

Within the methods of the present invention, if the nucleic acid sequence is an RNA sequence, it is preferred that the pre-determined RNA sequence is comprised in 16S rRNA or 23S rRNA, in particular the 16S rRNA or 23S rRNA of one of the species listed above, in particular M. orale, M. arginini, M. fermentans, M. hyorhinis, A. laidlawii, M. hominis, M. synoviae, S. citri, M. pneumoniae, M. bovis, M. salivarium or M. gallisepticum.

It is preferred that the primers used in the methods of the invention are some or preferably all of:

1. FIP primer comprises a sequence of
(SEQ ID NO: 1)
TCA TCG TTT ACA GCG TGG ACG AAA GCG TGG GGA GCA;
and/or
2. BIP primer comprises a sequence of
(SEQ ID NO: 2)
GCA GCT AAC GCA TTA AAT AGT TTC ACT CTT GCG AGC,
and/or
3. LPF primer comprises a sequence of
(SEQ ID NO: 3)
CTA CCA GGG TAT CTA ATC;
and/or
4. LPB primer comprises a sequence of
(SEQ ID NO: 4)
TGA TCC GCC TGA GTA GTA;
and/or
5. B3 primer comprises a sequence of
(SEQ ID NO: 5)
CGG GTC CCC GTC AAT TCC.

The above primer sequences target a sequence of Mycoplasma orale. In particular, the above primer target the following sequence

#AY796060.1_Mycoplasma_orale_strain_NC10112_16S_
ribosomal_RNA_gene_complete_Sequence
(SEQ ID NO: 7)
AGAGTTTGATCCTGGCTCAGGATGAACGCTGGCTGTGTGCCTAATACATG
CATGTCGAGCGGAAGTAGCAATACTTTAGCGGCGAATGGGTGAGTAACAC
GTGCTTAATCTACCTTTTAGATTGGAATACCTAATGGAAACATTGGTTAA
TGCCGGATACGCATGAAGTCGCATGACTTCGTTGTGAAAGGAGCGTTTGC
TCCGCTAAGAGATGAGGGTGCGGAACATTAGCTAGTTGGTGAGGTAATGG
CCCACCAAGGCTATGATGTTTAGCCGGGTCGAGAGACTGAACGGCCACAT
TGGGACTGAGATACGGCCCAAACTCCTACGGGAGGCAGCAGTAGGGAATA
TTCCACAATGAGCGAAAGCTTGATGGAGCGACACAGCGTGCACGATGAAG
GCCCTCGGGTTGTAAAGTGCTGTTGCAAGGGAAGAACAGTTAGTTGAGGA
AATGCTTCTAATCTGACGGTACCTTGTTAGAAAGCGACGGCTAACTATGT
GCCAGCAGCCGCGGTAATACATAGGTCGCAAGCGTTATCCGGAATTATTG
GGCGTAAAGCGTTCGTAGGCTGTTTATTAAGTCTGGAGTCAAATCCCAGG
GCTCAACCCTGGCTCGCTTTGGATACTGGTAAACTAGAGTTAGATAGAGG
TAAGCGGAATTCCATGTGGAGCGGTGAAATGCGTAGATATATGGAAGAAC
ACCAAAGGCGAAGGCAGCTTACTGGGTCTATACTGACGCTGAGGGACGAA
AGCGTGGGGAGCAAACAGGATTAGATACCCTGGTAGTCCACGCTGTAAAC
GATGATCATTAGTCGGTGGAAAACTACTGACGCAGCTAACGCATTAAATG
ATCCGCCTGAGTAGTATGCTCGCAAGAGTGAAACTTAAAGGAATTGACGG
GGACCCGCACAAGCGGTGGAGCATGTGGTTTAATTTGAAGATACGCGGAG
AACCTTACCCACTCTTGACATCCCCTGCAAAGCTATAGAGATATAGTAGA
GGTTAACAGGGTGACAGATGGTGCATGGTTGTCGTCAGCTCGTGTCGTGA
GATGTTTGGTCAAGTCCTGCAACGAGCGCAACCCCTATCTTTAGTTACTA
ACGAGTCATGTCGAGGACTCTAGAGATACTGCCTGGGTAACCGGGAGGAA
GGTGGGGATGACGTCAAATCATCATGCCTCTTACGAGTGGGGCTACACAC
GTGCTACAATGGTCGGTACAAAGAGAAGCAATATGGCGACATGGAGCAAA
TCTCAAAAAGCCGATCTCAGTTCGGATTGAAGTCTGCAATTCGACTTCAT
GAAGTCGGAATCGCTAGTAATCGCAGATCAGCTACGCTGCGGTGAATACG
TTCTCGGGTCTTGTACACACCGCCCGTCACACCATGGGAGCTGGTAATAC
CCAAAGTCGGTTTGCTAACCTCGGAGGCGACTGCCTAAGGTAGGACTGGT
GACTGGGGTGAAGTCGTAACAAGGTATCCCTACGAGAACGTGCGGCTGGA
TCACCTCCTT

In some embodiments, the primers used in the methods of the invention, in particular for Mycoplasma orale, comprise at least one selected from the group of:

a) a FIP primer comprising a sequence that has at least 88%, 91%, 94%, 97% or 100% sequence identity to the sequence: TCA TCG TTT ACA GCG TGG ACG AAA GCG TGG GGA GCA (SEQ ID NO: 1), which sequence still provides the primer functionality,

b) a BIP primer comprising a sequence that has at least 88%, 91%, 94%, 97% or 100% sequence identity to the sequence: GCA GCT AAC GCA TTA AAT AGT TTC ACT CTT GCG AGC (SEQ ID NO: 2), which sequence still provides the primer functionality,

c) a LPF primer comprising a sequence that has at least 88%, 94%, or 100% sequence identity to the sequence: CTA CCA GGG TAT CTA ATC (SEQ ID NO: 3), which sequence still provides the primer functionality,

d) a LPB primer comprising a sequence that has at least 88%, 94%, or 100% sequence identity to the sequence: TGA TCC GCC TGA GTA GTA (SEQ ID NO: 4), which sequence still provides the primer functionality, and

e) a B3 primer comprising a sequence that has at least 88%, 94%, or 100% identical to the sequence CGG GTC CCC GTC AAT TCC (SEQ ID NO: 5), which sequence still provides the primer functionality,

preferably wherein the primer functionality is primer functionality at the SEQ ID NO: 7.

“Percent (%) sequence identity” with respect to a reference sequence is defined as the percentage of nucleotides in a candidate sequence that are identical with the nucleotides in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

However, the skilled person is well-aware how to design alternative or further primer sequences depending on the target sequence to be detected in the sample (see e.g., Jia, B., et al., 2019, Frontiers in microbiology, 10, 2860).

Within the methods of the present invention, if the nucleic acid sequence is a DNA sequence, it is preferred that the pre-determined DNA sequence is comprised in the gene coding for the rRNA of a bacterium of the Mollicutes class.

The terms “rRNA”, as used herein, refer to the RNA that is the primary constituent of ribosomes. In general, there are two mitochondrial rRNA molecules (23S and 16S) and four types of cytoplasmic rRNA (28S, 5.8S, 5S and 18S).

Accordingly, within the methods of the present invention, if the nucleic acid sequence is a DNA sequence, it is preferred that the pre-determined DNA sequence is comprised in the gene coding for the 16S rRNA or 23S rRNA, in particular the 16S rRNA or 23S rRNA of one of the species listed above, in particular M. orale, M. arginini, M. fermentans, M. hyorhinis, A. laidlawii, M. hominis, M. synoviae, S. citri, M. pneumoniae, M. bovis, M. salivarium or M. gallisepticum.

The term “gene coding for 16S rRNA”, as used herein, refers to a DNA sequence from the relevant organism that allows encoding of 16S ribosomal RNA and may additionally include the 16S ribosomal intergenic region located between the 16 s ribosomal RNA gene and the 23S ribosomal RNA gene. In a certain embodiment of the invention the gene coding for 16S rRNA is SEQ ID NO: 7.

The term “gene coding for 23S rRNA” as used herein, refers to a DNA sequence from the relevant organism that allows encoding of 23S ribosomal RNA.

Within the present invention, the sample can be of any kind. However, it is preferred that the sample is obtained from primary- or modified cells and/or tissues, cell cultures, culture medium and/or additives, cell derived products, laboratory equipment, biopharmaceutical products such as advanced therapy medicinal products (ATMPs).

Cells can be modified, e.g. by genetic manipulation or passaging in culture, to achieve desired properties.

The terms “primary cell” or “primary culture” as used herein, refer to a cell or a culture of cells that have been explanted directly from an organism, organ, or tissue.

The term “cell culture”, as used herein refers to the maintenance, growth, propagation, or expansion of cells in an artificial in vitro environment outside of a multicellular organism or tissue. Typically, cell culture is performed under sterile, controlled temperature and atmospheric conditions in tissue culture plates (e.g., 10-cm plates, 96-well plates, etc.), or other adherent culture (e.g., on microcarrier beads) or in suspension culture such as in roller bottles. Cultures can be grown inter alia in petri-dishes, shake flasks, small scale bioreactors, and/or large-scale bioreactors. A bioreactor is a device used to culture cells in which environmental conditions such as temperature, atmosphere, agitation, and/or pH can be monitored, adjusted and controlled.

The term “culture medium”, as used herein, refers to a solid or a liquid substance used to support the maintenance, growth, propagation, and/or expansion of cells.

The term “cell derived products” refers to any product synthesized by the cell or a product made in the cultivation vessel using a product synthesized by the cell. The cell derived product also comprises a product generated in the cultivation vessel with the help of a cell component, e.g. a product converted from a substrate added to the cell culture by enzymes produced/derived from the cells. Cell derived products include (but are not limited to) proteins like growth factors, cytokines, monoclonal antibodies, immunoglobulin products, enzymes, hormones, fusion proteins, recombinant proteins, in particular proteins, which are secreted into the cell culture medium. Cell-derived products also comprise components derived from the cells, such as membranes, cell walls, organelles, proteins, enzymes, nucleic acids, ribosomes, pigments, primary and secondary metabolites. Moreover, cell-derived products also comprise cell extracts or fractions (mixtures containing any combination of the before mentioned components).

The term “biopharmaceutical product”, as used herein, refers to one or more product(s) obtained from biotechnology such as culture media, cellular cultures, buffer solutions, artificial nutrition liquids, blood products, and derivatives of blood products or a or more pharmaceutical product(s), or more generally a product that is designed to be used in the medical field. Such a product is in liquid, pasty or powder form, in one or more phases, homogeneous or not. The invention also applies to products other than biopharmaceutical products, according to the definition that was just given, but that are subject to analogous requirements relative to their processing.

The terms “advanced therapy medicinal products” or “ATMPs” refer to biopharmaceutical products comprising cellular material that is used for therapeutic purposes. ATMPs include, but are not limited to, gene therapy medicines, somatic-cell therapy medicines and tissue-engineered medicines.

Primary- or modified cells and/or tissues, cell cultures, culture medium and/or additives, cell derived products, laboratory equipment, biopharmaceutical products are particularly sensitive to bacterial contamination and bacteria of the Mollicutes class are among the most common contaminants.

Therefore, the method of the invention is particularly useful to determine contaminations in certain samples.

In the methods of the present invention, in particular in step (c) thereof, the temperature can be fixed.

The term “fixed temperature”, as used herein, refers to keeping the temperature condition constant or almost constant so that enzymes and primers can substantially function. The almost constant temperature condition means that not only the set temperature is accurately maintained but also a slight change in the temperature is acceptable within such a degree that it does not spoil substantial functions of the enzymes and primers. For example, a change in temperature of approximately from 0 to 10° C. is acceptable.

The nucleic acid amplification reaction under a fixed temperature can be carried out by keeping the temperature at such a level that activity of the enzyme to be used can be maintained. In addition, in order to effect annealing of a primer with the target nucleic acid in said nucleic acid amplification reaction, for example, to set the reaction temperature may be set to the temperature of around the Tm value of the primer or lower than that, and it is preferred to set it at a level of stringency by taking the Tm value of the primer into consideration. In said nucleic acid amplification reaction, the amplification reaction can be repeated until the enzyme is inactivated or one of the reagents including primers is used up.

That is, the one or more enzyme(s), DNA primers and the sample to be analyzed are incubated in the same tube at a constant temperature. The temperature is preferably between 50 and 75° C. However, the temperature may also be lower, for example between 30 and 75° C. In an alternative embodiment, a touchdown temperature step is used. That is, the temperature is lowered during the course of the analysis, for example starting at a temperature of 70° C. that is subsequently lowered to 50° C.

In the methods of the present invention, the one or more enzyme(s), DNA primers and the sample to be analyzed are incubated in the same tube for a time between 1 and 120 minutes, preferably between 1 and 60, 1 and 45, 1 and 30 or between 1 and 15 minutes. In a preferred embodiment, the sample is incubated for 3, 4, 5, 6, 7, 8, 9, 10, 11, 12, 13, 14, 15, 16, 17, 18, 19, 20, 21, 22, 23, 24, 25, 26, 27, 28, 29 or 30 minutes.

The methods of the present invention comprise a step of determining whether a double-stranded elongated DNA sequence is present in the sample, in particular wherein presence of the double-stranded elongated DNA sequence in the sample is indicative of the presence of the pre-determined nucleic acid sequence in the sample. The skilled person is well-aware of methods suitable to be used for determining presence of a double-stranded DNA sequence in a sample, in particular where the sequence to be detected is known. Thus, any method known to the skilled person for that purpose may be used within the present invention. However, it is preferred that the presence of the elongated double-stranded DNA is determined by using a nucleic acid molecule hybridisable to the elongated double-stranded DNA sequence, in particular wherein the nucleic acid molecule is labelled, using a molecule that intercalates in the elongated double-stranded DNA sequence or using turbidity measurement.

The term “label” or grammatical variations thereof, as used herein, refer to any detectable or signal-generating molecule or reporter molecule. Convenient labels include colorimetric, chemiluminescent, chromogenic, radioactive and fluorescent labels, but enzymatic (e.g. colorimetric, luminescent, chromogenic) or antibody-based labelling methods or signal-generating systems may also be used. Thus, the term “label” as used herein includes not only directly detectable signal-giving or passive moieties, but also any moiety which generates a signal or takes part in a signal generating reaction or that may be detected indirectly in some way. “labelled” as used herein, refers to being connected with or linked to a detectable label. Determining whether an elongated double-stranded DNA sequence is present in the sample may be achieved via fluorescence reporting. The majority of such approaches are based on the use of intercalating dyes, such as ethidium bromide, SYBR Green. EvaGreen and YO-PRO-I (Zhang X, et al. 2013, PLoS One 8(12):e82841; Mair G. et al, 2013, BMC Veterinary Research 9: 108.). As used herein, an agent or dye that “intercalates” refers to an agent or moiety capable of non-covalent insertion between stacked base pairs in a nucleic acid double helix. Determining whether an elongated double-stranded DNA sequence is present in the sample may be achieved by a Fluorescence technique that relies on the mechanism of Forster resonance energy transfer (FRET) (Chen Q, et al., 1997, Biochemistry 36(15):4701-11). In certain embodiments of the invention, the LPB and/or LPF are labelled at the 5′ end with at least one label and/or acceptor fluorophore.

The term “turbidity”, as used herein, refers to a measure of the suspended and/or soluble particles in a fluid or transparent solid that causes light to be scattered or absorbed. In certain embodiments of the invention, indirect determination of whether an elongated double-stranded DNA sequence is present in the sample relies essentially on the formation of pyrophosphate as a reaction byproduct. Pyrophosphate ions can be released by incorporation of deoxynucleotide triphosphates (dNTPs) into the DNA strand during nucleic acid polymerization and these ions react with divalent metal ions, particularly magnesium ions, present in the reaction mix to produce a white, insoluble magnesium pyrophosphate precipitate as described by Mori Y., et al. 2001 (Biochem. Biophys. Res. Commun. 289: 150-154). This participate results in a progressive increase in the turbidity of the reaction solution and pyrophosphate precipitates can be measured quantitatively in terms of turbidity or observed by the naked eye as a pellet after centrifugation. In an alternative embodiment of the invention, determining whether an elongated double-stranded DNA sequence is present in a sample is achieved through the incorporation of manganese ions and calcein in the reaction. Calcein's fluorescence is naturally quenched by binding of manganese ions. Pyrophosphate production as a reaction byproduct removes manganese ions form the buffer through precipitation, and the increased turbidity coupled with restored calcein fluorescence enables an easy visual read-out upon excitation with either visible or UV light (Tomita N., et al, 2008. Nat. Protoc. 3:877-882). In still another embodiment of the invention, the enzymatic conversion of pyrophosphate into ATP, which is produced during DNA synthesis, is monitored through the bioluminescence generated by thermostable firefly luciferase for determining whether an elongated double-stranded DNA sequence is present in the sample (Gandelman O A., et al. 2010, PLoS One 5(11): e14155). Generally, all methods described by Becherer, Lisa, et al, (“Loop-mediated isothermal amplification (LAMP)—review and classification of methods for sequence-specific detection.” Analytical Methods 12.6 (2020): 717-746) can be combined with the method of the invention.

In a further embodiment, the present invention relates to a method of decontaminating a cell and/or tissue culture, infected by a bacterium of the Mollicutes class, the method comprising administering to the culture an efficient amount of an antibiotic, wherein the culture has previously been determined to be infected by a bacterium of the Mollicutes class using the method of the present invention. Preferably, the antibiotic drug is BM Cyclin.

The term “efficient amount”, as used herein, refers to the amount of an active agent (such as one or more compounds provided herein alone, in combination, or potentially in combination with other agent(s)) sufficient to induce a desired biological result.

The terms “antibiotic”, as used herein, refers to an agent, a drug or a composition with properties useful against bacteria and/or in the treatment of bacteria-related disease. The antibiotic may have, inter alia, properties of preventing, inhibiting, suppressing, reducing, adversely impacting, and/or interfering with the growth, survival, replication, function, and/or dissemination of a bacterium. Any antibiotic can be used in the context of the invention. However, some antibiotics are particularly useful in that the bacteria of the Mollicutes class do not tend to develop resistance against these compounds (Vladislav M Chernov, et al., 2018, FEMS Microbiology Letters, Volume 365, Issue 18, fny185). In some embodiments, the antibiotic comprises at least one selected from the group of tetracycline, fluoroquinolone, macrolide, BM Cyclin and inhibitor of deformylase.

The term “BM Cyclin” refers to a composition for the elimination of mycoplasma from infected cell cultures without obvious marked cytotoxic side effects. BM Cyclin typically comprises Tiamulin and Minocyclin.

The method of the invention can efficiently determine the bacterium of the Mollicutes class and facilitates early detection, screening, monitoring. Therefore, the method of the invention improves the specific use of antibiotics, e.g., in decontamination procedures.

In order to carry out the method of the invention, the kit can be prepared by collecting necessary reagents. In a further embodiment, the invention relates to a kit, in particular a kit for use in detecting a nucleic acid molecule in a sample, in particular detecting contamination with a bacterium of the Mollicutes class in a sample. The kit comprises one or more, preferably all five or six primers for detecting a pre-determined sequence of the bacterium of the Mollicutes class. The kit may also comprise more than one primer system, in particular two or more primer systems targeting different sequences of the same bacterium or different bacteria of the Mollicutes class. As such, the methods of the invention and the kit of the invention can be used to detect more than one different bacterium of the Mollicutes class in a sample by using more than one primer system, e.g. by using primers that contain a quencher-fluorophore duplex region (Tanner N A, Zhang Y, Evans T C Jr. Simultaneous multiple target detection in real-time loop-mediated isothermal amplification. Biotechniques. 2012; 53(2):81-89.). In this regard, it was surprisingly found that the reduced number of primers leads to an improved usability of more than one primer systems due to reduced primer interference.

In a particularly preferred embodiment of the present invention, the kits (to be prepared in context) of this invention or the methods and uses of the invention may further comprise or be provided with (an) instruction manual(s). For example, said instruction manual(s) may guide the skilled person (how) to employ the kit of the invention in the diagnostic uses provided herein and in accordance with the present invention. Particularly, said instruction manual(s) may comprise guidance to use or apply the herein provided methods or uses.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The general methods and techniques described herein may be performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990).

While aspects of the invention are illustrated and described in detail in the figures and foregoing description, such illustration and description are to be considered illustrative or exemplary and not restrictive. It will be understood that changes and modifications may be made by those of ordinary skill within the scope and spirit of the following claims. In particular, the present invention covers further embodiments with any combination of features from different embodiments described above and below.

FIG. 1 shows a comparison of the five and six primer system for detecting a bacterium of the Mollicutes class.

Furthermore, in the claims the word “comprising” does not exclude other elements or steps, and the indefinite article “a” or “an” does not exclude a plurality. A single unit may fulfill the functions of several features recited in the claims. The terms “essentially”, “about”, “approximately” and the like in connection with an attribute or a value particularly also define exactly the attribute or exactly the value, respectively. Any reference signs in the claims should not be construed as limiting the scope.

### EXAMPLES

The following are examples of methods and compositions of the invention. It is understood that various other embodiments may be practiced, given the general description provided above.

The Novel 5 Primer System without F3 Amplifies Mollicutes as Efficient as 6 Primer System with F3

TABLE 1
Primers

| | | | |
|---|---|---|---|
| FIP | TCA TCG TTT ACA GCG TGG ACG AAA | LPF | CTA CCA GGG TAT CTA ATC |
| | GCG TGG GGA GCA (SEQ ID NO: 1) | | (SEQ ID NO: 3) |
| BIP | GCA GCT AAC GCA TTA AAT AGT TTC | LPB | TGA TCC GCC TGA GTA GTA |
| | ACT CTT GCG AGC (SEQ ID NO: 2) | | (SEQ ID NO: 4) |
| B3 | CGG GTC CCC GTC AAT TCC | F3 | CTA TAC TGA CGC TGA GGG |
| | (SEQ ID NO: 5) | | (SEQ ID NO: 6) |

TABLE 2
Primer mix: novel 5 primer system

| | Final concentration. |
|---|---|
| FIP | 1.6 μM |
| BIP | 1.6 μM |
| LPF | 0.8 μM |
| LPB | 0.8 μM |
| B3 | 0.4 μM |

TABLE 3
Primer mix: LAMP 6 primer system

| | Final concentration |
|---|---|
| FIP | 1.6 μM |
| BIP | 1.6 μM |
| LPF | 0.8 μM |
| LPB | 0.8 μM |
| B3 | 0.2 μM |
| F3 | 0.2 μM |

TABLE 4
Primer/Enzyme mix (PEM)

| | Vol/rx |
|---|---|
| Isothermal master mix | 15.0 μl |
| Primer mix | 2.0 μl |
| | 17.0 μl |

Add 17.0 μl PEM per reaction

Template Addition

Add 8.0 μl extracted RNA

Add 8.0 μl RNase-free H₂O as negative assay control

TABLE 5
Settings for isothermal amplification and dye acquisition

| | | Temper- | Acquisi- | | Ramp |
|---|---|---|---|---|---|
| | Cycles | ature | tion | Time | rate |
| Ampli- | 25 | 65° C. | None | 27 s | 4.4° C. |
| fication | | | Single | 30 s | 4.4° C. |
| | | | Quant | Melt | Integration |
| | Channel | Dye | Factor | Factor | Time |
| Dye | #1,470/ | SYBR | 20.00 | 1.2 | Dynamic |
| acquisition | 514 | Green I | | | |

1. A method for determining presence of a pre-determined nucleic acid sequence in a sample, the method comprising the steps of:
(a) adding one or more enzyme(s) providing activities of RNA- and/or DNA-dependent DNA polymerase activity and strand-displacement activity to the sample to be analysed for the presence of the pre-determined nucleic acid sequence;
(b) adding at least five DNA primers to the sample to be analysed for the presence of the pre-determined nucleic acid sequence, wherein at least one DNA primers comprises a sequence hybridisable to the nucleic acid sequence and at least one DNA primers comprise a sequence hybridisable to the DNA sequence reverse-complementary to the nucleic acid sequence;
(c) incubating the sample resulting from steps (a) and (b) at a fixed temperature;
(d) determining whether a double-stranded elongated DNA sequence is present in the sample,
wherein presence of the double-stranded elongated DNA sequence in the sample is indicative of the presence of the pre-determined nucleic acid sequence in the sample
wherein the pre-determined nucleic acid sequence is of a bacterium of the Mollicutes class and wherein no F3 primer is used.

2. The method of claim 1, wherein the at least five primers comprise a forward inner primer (FIP), backward inner primer (BIP), loop primer forward (LPF) and loop primer backwards (LPB), respectively.

3. The method of claim 1 or 2, wherein the at least five primers further comprise a B3 primer.

4. The method of any one of claims 1 to 3, wherein the pre-determined nucleic acid sequence is an RNA or DNA sequence.

5. The method of any one of claims 1 to 5, wherein the bacterium of the Mollicutes class is of the genus Mycoplasma, Spiroplasma, Acholeplasma, or Ureaplasma.

6. The method of claim 6, wherein the bacterium is M. orale, M. arginini, M. fermentans, M. hyorhinis, A. laidlawii, M. hominis, M. synoviae, S. citri, M. pneumoniae, M. bovis, M. sahvarium or M. gallisepticum.

7. The method of any one of claims 4 to 6, wherein the RNA is comprised in 16S rRNA or 23S rRNA.

8. The method of any one of claims 4 to 6, wherein the DNA is the gene coding for 16S rRNA or the gene coding for 23 S rRNA.

9. The method of any one of claims 1 to 8, wherein the sample is obtained from primary- or modified cells and/or tissues, cell cultures, culture medium and/or additives, cell derived products, laboratory equipment or biopharmaceutical products such as ATMPs.

10. The method of any one of claims 1 to 9, wherein the fixed temperature is between 50 and 75° C.

11. The method of any one of claims 1 to 10, wherein the sample in step (c) is incubated for 1 to 120 minutes.

12. The method of any one of claims 1 to 11, wherein presence of the double-stranded elongated DNA sequence in the sample is determined by using a nucleic acid molecule hybridisable to the double-stranded elongated DNA sequence, in particular wherein the nucleic acid molecule is labelled, using a molecule that intercalates in the double-stranded elongated DNA sequence or using turbidity measurement.

13. A method of decontaminating a cell and/or tissue culture infected by a bacterium of the Mollicutes class, the method comprising administering to the culture an efficient amount of an antibiotic, wherein the culture has previously been determined to be infected by a bacterium of the Mollicutes class using the method of any one of claims 1 to 12.

14. The method of claim 13, wherein the antibiotic drug is BM Cyclin.


SEQUENCE LISTING (7 sequences)

SEQ ID NO: 1 - DNA, 36 bp, Artificial Sequence
  FIP primer
tcatcgtttacagcgtggacgaaagcgtggggagca

SEQ ID NO: 2 - DNA, 36 bp, Artificial Sequence
  BIP primer
gcagctaacgcattaaatagtttcactcttgcgagc

SEQ ID NO: 3 - DNA, 18 bp, Artificial Sequence
  LPF primer
ctaccagggtatctaatc

SEQ ID NO: 4 - DNA, 18 bp, Artificial Sequence
  LPB primer
tgatccgcctgagtagta

SEQ ID NO: 5 - DNA, 18 bp, Artificial Sequence
  B3 primer
cgggtccccgtcaattcc

SEQ ID NO: 6 - DNA, 18 bp, Artificial Sequence
  F3 primer
ctatactgacgctgaggg

SEQ ID NO: 7 - DNA, 1510 bp, Artificial Sequence
  Mycoplasma orale strain NC10112 16S ribosomal RNA gene
agagtttgatcctggctcaggatgaacgctggctgtgtgcctaatacatgcatgtcgagcggaagtagcaatactttagcggcgaatgggtgagtaacacgtgcttaatctaccttttagattggaatacctaatggaaacattggttaatgccggatacgcatgaagtcgcatgacttcgttgtgaaaggagcgtttgctccgctaagagatgagggtgcggaacattagctagttggtgaggtaatggcccaccaaggctatgatgtttagccgggtcgagagactgaacggccacattgggactgagatacggcccaaactcctacgggaggcagcagtagggaatattccacaatgagcgaaagcttgatggagcgacacagcgtgcacgatgaaggccctcgggttgtaaagtgctgttgcaagggaagaacagttagttgaggaaatgcttctaatctgacggtaccttgttagaaagcgacggctaactatgtgccagcagccgcggtaatacataggtcgcaagcgttatccggaattattgggcgtaaagcgttcgtaggctgtttattaagtctggagtcaaatcccagggctcaaccctggctcgctttggatactggtaaactagagttagatagaggtaagcggaattccatgtggagcggtgaaatgcgtagatatatggaagaacaccaaaggcgaaggcagcttactgggtctatactgacgctgagggacgaaagcgtggggagcaaacaggattagataccctggtagtccacgctgtaaacgatgatcattagtcggtggaaaactactgacgcagctaacgcattaaatgatccgcctgagtagtatgctcgcaagagtgaaacttaaaggaattgacggggacccgcacaagcggtggagcatgtggtttaatttgaagatacgcggagaaccttacccactcttgacatcccctgcaaagctatagagatatagtagaggttaacagggtgacagatggtgcatggttgtcgtcagctcgtgtcgtgagatgtttggtcaagtcctgcaacgagcgcaacccctatctttagttactaacgagtcatgtcgaggactctagagatactgcctgggtaaccgggaggaaggtggggatgacgtcaaatcatcatgcctcttacgagtggggctacacacgtgctacaatggtcggtacaaagagaagcaatatggcgacatggagcaaatctcaaaaagccgatctcagttcggattgaagtctgcaattcgacttcatgaagtcggaatcgctagtaatcgcagatcagctacgctgcggtgaatacgttctcgggtcttgtacacaccgcccgtcacaccatgggagctggtaatacccaaagtcggtttgctaacctcggaggcgactgcctaaggtaggactggtgactggggtgaagtcgtaacaaggtatccctacgagaacgtgcggctggatcacctcctt